SEQUENCE LISTING (1 sequence)

SEQ ID NO: 1 - protein, 65 aa, Homo sapiens
ITCPPPMSVEHADIWVKSYSLYSRERYICNSGFKRKAGTSSLTECVLNKATNVAHWTTPSLKCIR

(19) United States
(12) Patent Application Publication
(10) Pub. No.: US 2025/0312418 A1
(43) Pub. Date: Oct. 9, 2025

(54) INTERLEUKIN-15 SUPERAGONIST N-803 IN PEOPLE EXPERIENCING LONG COVID

(71) Applicant: ImmunityBio, Inc., Culver City, CA (US)

(72) Inventors: Patrick Soon-Shiong, Culver City, CA (US); Jeffrey T. Safrit, Culver City, CA (US)

(21) Appl. No.: 19/173,198

(22) Filed: Apr. 8, 2025

Related U.S. Application Data
(60) Provisional application No. 63/631,924, filed on Apr. 9, 2024.

(51) Int. Cl. A61K 38/20; A61K 9/00; A61K 38/17; A61K 39/215; A61P 31/14; C07K 16/28

(52) U.S. Cl. CPC A61K 38/2086; A61K 9/0019; A61K 38/1793; A61K 39/215; A61P 31/14; C07K 16/283

(57) ABSTRACT

Disclosed herein are methods of treating one or more symptoms associated with a viral infection in a patient exposed to a virus. wherein the virus is no longer detectable in the patient, and wherein the symptoms develop or persist after clearance of the virus. The methods comprise administering to the patient an effective amount of a pharmaceutical composition comprising interleukin-15 (IL-15) or an IL-15 agonist derivative thereof.

### CROSS-REFERENCE TO RELATED APPLICATIONS

This application claims the benefit of priority under 35 U.S.C. § 119(e) to U.S. Provisional Patent Application No. 63/631,924, filed Apr. 9, 2024. The entire disclosure of which is incorporated herein by reference in its entirety.

### REFERENCE TO SEQUENCE LISTING

This application contains a Sequence Listing submitted electronically as an XML file and is hereby incorporated by reference in its entirety. Said XML file, created on Apr. 7, 2025, is named 8774_83_seq_lisitng.xml and is 1,952 bytes in size.

### BACKGROUND

Coronavirus disease 2019 (COVID-19) is caused by Severe Acute Respiratory Coronavirus type 2 (SARS-CoV2), a ribonucleic acid (RNA) virus which emerged in Wuhan, China, in December 2019 and spread across the world at an unprecedented pace through human-to-human transmission. The spike protein of the 2019 novel coronavirus is a surface protein which binds to angiotensin-converting enzyme-2 (ACE-2) on human cells. The S1 subunit catalyzes attachment to ACE-2 and the S2 subunit allows fusion with cell membranes and subsequent entry into the cell. As a result, the spike protein is a relevant target for drug development. Moreover, antibodies (Abs) against the spike protein are able to neutralize the virus, prevent infection, and reduce the severity of disease.

The COVID-19 pandemic has resulted in a growing population of individuals recovering from SARS-CoV2 infection. Approximately one in five American adults who have had COVID-19 still have symptoms of Long COVID (LC), a type of post-acute sequelae of SARS-CoV2 infection (PASC) (Robertson MM, et al. The Epidemiology of Long Coronavirus Disease in US Adults. Clin Infect Dis 2023;76:1636-45). Some aspects of this recovery may be unique to COVID-19, but many appear to be similar to recovery from other viral illnesses, critical illnesses, and/or sepsis (Nalbandian A, et al. Post-acute COVID-19 syndrome. Nat Med 2021). LC is comprised of a broad range of symptoms that develop during or after COVID-19, continue for ≥2 months (i.e., three months from the onset of illness), have an impact on the patient's life, and are not explained by an alternative diagnosis. Consensus around the clinical definition of LC has progressed significantly, and as of Oct. 1, 2021, there has been an International Classification of Diseases, Tenth Revision, Clinical Modification, (ICD-10), for unspecified post-COVID conditions (U09.9). The World Health Organization (WHO) has also created a global COVID-19 clinical platform CRF for clinicians and patients to collect and report information (Global Burden of Disease Long CC, Wulf Hanson S, Abbafati C, et al. Estimated Global Proportions of Individuals With Persistent Fatigue, Cognitive, and Respiratory Symptom Clusters Following Symptomatic COVID-19 in 2020 and 2021. JAMA 2022;328:1604-15). The United States (US) Department of Health and Human Services (HHS) and the Department of Justice (DoJ) released a guidance statement on LC as a disability under the Americans with Disabilities Act, the Rehabilitation Act of 1973, and the Patient Protection and Affordable Care Act.

Persistent viral infection with viral reservoirs after the initial acute illness is one potential pathogenic mechanism for LC (Proal D, et al. SARS-CoV2 reservoir in post-acute sequelae of COVID-19 (PASC). Nat Immunol 2023;24:1616-2). Theoretically, virus persistence may be driving inflammation and clotting, leading to the diffuse tissue damage and the symptoms which are associated with LC. If this model is correct, then purging persistent viral reservoirs will result in reduced tissue damage and resolution of symptoms. Recent anecdotal experience with readily available antiviral drugs (e.g., PAXLOVID™) in people with LC provide support for this concept (Peluso M J, et al. Effect of Oral Nirmatrelvir on Long COVID Symptoms: 4 Cases and Rationale for Systematic Studies. Pathog Immun 2022;7:95-103). The finding that LC is less common in people who were vaccinated prior to infection is also indirect evidence for this model. There is still an unmet need for treating individuals with LC. As provided herein, the safety and tolerability of subcutaneous administration of an IL-15 superagonist (N-803) in individuals who are experiencing Long COVID is demonstrated.

### SUMMARY

One embodiment relates to a method of treating one or more symptoms associated with a viral infection in a patient exposed to a virus, wherein the virus is no longer detectable in the patient, and wherein the symptoms develop or persist after clearance of the virus, the method comprising administering to the patient an effective amount of a pharmaceutical composition comprising interleukin-15 (IL-15) or an IL-15 agonist derivative thereof.

One embodiment relates to the use of a pharmaceutical composition comprising IL-15 or an IL-15 agonist derived thereof, for treating one or more symptoms associated with a viral infection in a patient wherein the virus is no longer detectable in the patient, and wherein the symptoms develop or persist after clearance of the virus.

In one aspect, the symptoms are maintained for longer than 4 weeks after the patient has been diagnosed with the viral infection. In one aspect, the viral infection is acute SARS-CoV2 infection.

In still another aspect, the symptoms are maintained for longer than 12 weeks after the patient has been diagnosed with the viral infection.

In yet another aspect, the symptoms are maintained for longer than 24 weeks after the patient has been diagnosed with the viral infection.

In one aspect, the symptoms are selected from the group consisting of neurological symptoms, cardiovascular symptoms, cardiopulmonary symptoms, gastro-intestinal symptoms, musculoskeletal symptoms, systemic symptoms and combinations thereof.

In one aspect, the symptoms are neurological symptoms. In one aspect, the neurological symptoms are selected from the group consisting of fatigue, sleep disturbance, headache, cognitive impairment, memory loss, attention disorder, post-traumatic stress disorder (PTSD), anxiety, depression and combinations thereof.

In one aspect, the symptoms are cardiovascular symptoms. In one aspect, the cardiovascular symptoms are selected from the group consisting of hypertension, cardiac dysrhythmias, poor circulation, coronary atherosclerosis, heart failure and combinations thereof.

In one aspect, the symptoms are gastro-intestinal symptoms. In one aspect, the gastro-intestinal symptoms are selected from the group consisting of loss of appetite, nausea, acid reflux, diarrhea, abdominal distension, belching, vomiting, abdominal pain, bloody stools and combinations thereof.

In one aspect, the symptoms indicate the patient has an endocrine disorder and/or a metabolic disorder and/or a pulmonary disorder.

In still another aspect, the virus is a coronavirus. In one aspect, the virus is SARS-CoV2. In one aspect the SARS-CoV2 virus causes COVID-19. In one aspect, patient recovery from acute SARS-CoV2 infection is prolonged. In one aspect, patient positivity for SARS-CoV2 infection exceeds 14 days. In one aspect, the patient has been diagnosed with long COVID (LC).

In still another aspect, the patient has post-viral fatigue.

In yet another aspect, the patient has dyspnea.

In one aspect, the patient has exacerbated chronic disease.

In one aspect, a non-specific immunoglobulin G (IgG) or fragment crystallizable (Fc) fragment thereof is co-administered to the patient, wherein the IgG or Fc fragment binds to CD16 on the patient's natural killer (NK) cells.

In one aspect, NK cell-mediated antibody dependent cellular cytotoxicity (ADCC) is inhibited.

In still another aspect, the IL-15 agonist derivative is a stabilized fusion construct.

In yet another aspect, the IL-15 agonist derivative is IL-15: IL-15Ra.

In one aspect, the IL-15 agonist derivative is nogapendekin alpha imbakicept (N-803).

In still another aspect, the IL-15 or IL-15 agonist derivative thereof is administered subcutaneously.

In yet another aspect, the IL-15 or IL-15 agonist derivative thereof is administered to the abdomen.

In one aspect, the IL-15 or IL-15 agonist derivative thereof is administered every 14 days. In one aspect, the IL-15 or IL-15 agonist derivative thereof is administered two or more times.

In yet another aspect, the IL-15 or IL-15 agonist derivative thereof is administered at 1 μg/kg to 50 μg/kg body weight. In still another aspect, the IL-15 or IL-15 agonist derivative thereof is administered at 10 μg/kg body weight.

In still another aspect, the patient has at least two symptoms. In one aspect, the two symptoms have been present for at least 60 days.

In yet another aspect, the method further comprises administering a SARS-CoV2 vaccine. In one aspect, the SARS-CoV2 vaccine comprises an Ad5[E1-, E2b-] adenoviral vector comprising a nucleic acid sequence encoding a SARS-CoV2 S protein and a nucleic acid sequence encoding a SARS-CoV2 nucleocapsid protein (N) protein fused to an ETSD sequence. In one aspect, the SARS-CoV2 vaccine is administered as a prime and a boost vaccination.

In still another aspect, the patient reports improvement in symptoms.

In one aspect, detection of SARS-COV2 plasma remnants is reduced and/or eliminated following administration of the IL-15 or IL-15 agonist derivative thereof. In one aspect, the IL-15 agonist derivative is N-803.

In one aspect, the patient has increased NK cells frequencies following administration of the IL-15 or IL-15 agonist derivative thereof. In one aspect, the IL-15 agonist derivative is N-803.

In yet another aspect, the patient has increased SARS-COV2 specific CD4 and CD8+ T cell responses following administration of the IL-15 or IL-15 agonist derivative thereof. In one aspect, the IL-15 agonist derivative is N-803.

In still another aspect, the patient has reduced SARS-COV2 RNA in gut tissue following administration of the IL-15 or IL-15 agonist derivative thereof. In one aspect, the IL-15 agonist derivative is N-803.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

Early in the pandemic, the common assumption was that SARS-CoV2 would prove to be a transient infection, as is the case with coronaviruses in general. This assumption was challenged by early reports that viral nucleic acid and proteins could be detected in the gut mucosa months after infection (Gaebler C, et al. Evolution of antibody immunity to SARS-CoV2. Nature 2021). Case reports emerged indicating that immunocompromised individuals including those with advanced malignancy, Human immunodeficiency virus (HIV)/Acquired Immunodeficiency Syndrome (AIDS), or on immunosuppression for autoimmune conditions, can harbor active replicating virus for many months (Aydillo T, et al. Shedding of Viable SARS-CoV2 after Immunosuppressive Therapy for Cancer. N Engl J Med 2020;383:2586-8; Choi B, et al. Persistence and Evolution of SARS-CoV2 in an Immunocompromised Host. N Engl J Med 2020; 383:2291-3; Zollner A, et al. Postacute COVID-19 is Characterized by Gut Viral Antigen Persistence in Inflammatory Bowel Diseases. Gastroenterology 2022;163:495-506.e8; Natarajan A, et al. Gastrointestinal symptoms and fecal shedding of SARS-CoV2 RNA suggest prolonged gastrointestinal infection. Med (N Y) 2022). More recently, similar observations have been made in immunocompetent people (Natarajan A, et al. 2022; Xu Q, et al. Adaptive immune responses to SARS-CoV2 persist in the pharyngeal lymphoid tissue of children. Nat Immunol 2023;24:186-99; Cheung CCL, et al. Residual SARS-CoV2 viral antigens detected in GI and hepatic tissues from five recovered patients with COVID-19. Gut 2022;71:226-9; Goh D, et al. Case report: Persistence of residual antigen and RNA of the SARS-CoV2 virus in tissues of two patients with long COVID. Front Immunol 2022; 13:939989; Stein S R, et al. SARS-CoV-2 infection and persistence in the human body and brain at autopsy. Nature 2022;612:758-63; Peluso M J, et al. Multimodal Molecular Imaging Reveals Tissue-Based T Cell Activation and Viral RNA Persistence for Up to 2 Years Following COVID-19. medRxiv 2023). Autopsy studies of people post-COVID dying from related or unrelated reasons also began to report presence of viral nucleic acid or protein in various tissues months after the infection was apparently cleared (Stein S R, et al. Nature 2022). Another provocative study demonstrated SARS-CoV2 in neonatal stool following remote maternal COVID-19 (Jin J C, et al. SARS CoV-2 detected in neonatal stool remote from maternal COVID-19 during pregnancy. Pediatr Res 2022:1-8). Taken together these studies provide growing support for SARS-CoV2 persistence.

SARS-CoV2 can infect several cell types and in theory any infected cell is at risk of harboring persistent virus. The precise localization of SARS-CoV2 persistence is unknown, but it is widely assumed that this occurs in tissues. This may or may not include immune-privileged sites as has been observed with other ribonucleic acid (RNA) viruses (e.g. Ebola) (Racine T, et al. Viral pathogenesis: Unlocking Ebola persistence. Nat Microbiol 2017;2:17124). Because tissue studies are generally impractical due to invasiveness of these procedures, there are now efforts to develop less-invasive measures to assess this. Some studies identified detectable SARS-CoV2 RNA in plasma and stool during the early post-acute phase (Tejerina F, et al. Post-COVID-19 syndrome. SARS-CoV2 RNA detection in plasma, stool, and urine in patients with persistent symptoms after COVID-19. BMC Infect Dis 2022;22:211). One recent study found that a large proportion of individuals with LC had at least intermittently detectable circulating antigen in the plasma for up to a year post-infection (Swank Z, et al. Persistent circulating SARS-CoV2 spike is associated with post-acute COVID-19 sequelae. Clin Infect Dis 2022). However, it remains unclear whether antigens can also persist in asymptomatic individuals, whether what is being detected represents remnants of a long extinguished infection or ongoing virus production from a long-lived reservoir or ongoing replication.

Data that SARS-CoV2 persists in gut lamina propria tissue in myeloid immune and other non-epithelial cells (Peluso MJ, et al. Multimodal Molecular Imaging Reveals Tissue-Based T Cell Activation and Viral RNA Persistence for Up to 2 Years Following COVID-19. medRxiv 2023, PMID: 37577714) has recently been generated by the inventors. Specifically, single-stranded SARS-CoV2 Spike RNA in 5 of 5 people with LC symptoms up to 1.8 years after initial infection and double stranded Spike RNA in 3 of these participants was identified. Double stranded RNA is only produced during viral replication or active viral life cycling and was observed in clusters of cells in the lamina propria and may represent microfoci of SARS-CoV2 persistence that has evaded immune cell-medicated clearance. Supporting this finding, people with LC have a dysregulated adaptive immune response with evidence of exhaustion of virus-specific CD8+ T cells (Yin, K., et al. Long COVID manifests with T cell dysregulation, inflammation and an uncoordinated adaptive immune response to SARS-CoV2; Nat. Immunol. 2024 Feb;25(2):218-225, PMID: 38212464). NK cells in people with severe acute COVID-19 tend to be exhausted with suboptimal cytotoxic function and cytokine production capacity while SARS-CoV2 infection in cells can lead to modulation of aberrant NK cell responses (Lee, M. J., et al., Defining the role of natural killer cells in COVID-19; Nat. Immunol. 2023 Oct; 24(10):1628-1638, PMID: 37460639). For example, SARS-CoV2 can escape NK cell killing through Nsp1-mediated downregulation of NKG2D ligands (Lee, M. J., et al. SARS-CoV2 escapes direct NK cell killing through Nsp1-mediated downregulation of ligands for NKG2D, Cell Rep. 2022 Dec.27;41(13):111892, PMID: 36543165). Whereas NK cell phenotype and function have not been extensively studied in the setting of LC, it is possible that chronic inflammation and NK cell exhaustion may facilitate chronic virus persistence that has been observed in tissues in several studies (Proal D, et al. SARS-CoV2 reservoir in post-acute sequelae of COVID-19 (PASC). Nat Immunol 2023;24:1616-27, PMID: 37667052). Modest elevations in inflammatory markers such as interleukin-6 (IL-6) and tumor necrosis factor alpha (TNF-α) have been identified in LC (Peluso M J, et al. Markers of Immune Activation and Inflammation in Individuals With Postacute Sequelae of Severe Acute Respiratory Syndrome Coronavirus 2 Infection. J Infect Dis 2021 Dec. 1;224(11):1839-1848, PMID: 34677601). While the use of an immunotherapy, like N-803, that transiently increases immune cell activation may lead to temporary exacerbation of LC symptoms and systemic inflammation may seem contradictory, purging viral reservoirs may lead to long-term reduction in symptoms and return to pre-COVID-19 immune homeostasis and general health. N-803 has been used safely in the setting of chronic HIV-1 infection, which is also associated with elevations of IL-6 and other cytokines (Sereti, I., et al. Persistent, Albeit Reduced, Chronic Inflammation in Persons Starting Antiretroviral Therapy in Acute HIV Infection, Clin. Infect. Dis. 2017 Jan. 15;64(2):124:131, PMID: 27737952).

It is anticipated by the inventors that tissue viral persistence contributes to LC pathophysiology, and that chronic viral persistence is the result of inadequate and/or dysregulated cytotoxic CD8+ T cell and NK responses. Furthermore, it is anticipated that enhancing effector immune function purges residual infection, thereby leading to longer-term reductions in immune activation, inflammation and return to pre-COVID-19 health.

Disclosed herein is a method of treating one or more symptoms associated with a viral infection in a patient exposed to a virus, wherein the virus is no longer detectable in the patient, and wherein the symptoms develop or persist after ostensible “clearance” of the virus. As provided herein the method comprises administering to the patient an effective amount of a pharmaceutical composition comprising IL-15 or an IL-15 agonist derivative thereof.

Further disclosed herein is the use of a pharmaceutical composition comprising IL-15 or an IL-15 agonist derived thereof, for treating one or more symptoms associated with a viral infection in a patient wherein the virus is no longer detectable in the patient, and wherein the symptoms develop or persist after clearance of the virus.

In one aspect, the symptoms associated with the viral infection are maintained or present for a period of at least 4 weeks after the patient has been diagnosed with the viral infection. In one aspect, the symptoms are maintained for a period of longer than 4 weeks, 5 week, 6 weeks, 7 weeks, 8 weeks, 9 weeks, 10 weeks, 11 weeks, 12 weeks, 13, weeks, 14 weeks, 15 weeks, 16 weeks, 17 weeks, 18 weeks, 19 weeks, 20 weeks, 21 weeks, 22 weeks, 23 weeks, 24 weeks, 25 weeks, or more than 25 weeks after the patient has been diagnosed with the viral infection. In one aspect, symptoms are maintained or present for at least 30 days, 35 days, 40 days, 45 days, 50 days, 55 days, 60 days, or more than 60 days after the patient has been diagnosed with the viral infection.

The symptoms caused by and/or associated with the viral infection (including SARS-CoV2 viral infection), include but are not limited to neurological symptoms, cardiovascular symptoms, cardiopulmonary symptoms, gastro-intestinal symptoms, musculoskeletal symptoms, systemic symptoms, and combinations thereof. In one aspect, the symptoms indicate the patient has an endocrine or metabolic disorder and/or a pulmonary disorder.

Neurological symptoms include fatigue, sleep disturbance, headache, cognitive impairment, memory loss, attention disorder, post-traumatic stress disorder (PTSD), anxiety, depression, dizziness, unsteadiness, neuropathy, dysautonomia/postural orthostatic tachycardia syndrome (POTS) and combinations thereof.

Cardiovascular symptoms include hypertension, cardiac dysrhythmia, poor circulation, coronary atherosclerosis, heart failure and combinations thereof.

Cardiopulmonary symptoms include chest pain, palpitations, shortness of breath, cough, fainting spells and combinations thereof.

Gastro-intestinal symptoms include loss of appetite, nausea, acid reflux, diarrhea, abdominal distension, belching, vomiting, abdominal pain, bloody stools and combinations thereof.

Musculoskeletal symptoms include muscle aches, joint pain and combinations thereof.

Systemic symptoms include fatigue, chills, post-exertional malaise and combinations thereof.

In one aspect, the virus that the patient has been exposed to is a coronavirus. In a preferred aspect, the coronavirus is SARS-CoV2.

In one aspect, the viral infection is acute SARS-CoV2 infection. In one aspect, the patient positivity for SARS-CoV2 viral infection exceeds 14 days. In still another aspect, the patient has post-viral fatigue, dyspnea and/or exacerbated chronic disease.

Patients diagnosed as having acute SARS-CoV2 viral infection wherein symptoms associated with the SARS-CoV2 viral infection have been present in the patient for at least 60 days (or greater than 2 months) are considered to have Long Covid (LC). In one aspect, the patient has at least two symptoms caused by and/or associated with the viral infection.

### Interleukin-15 (IL-15)

An “interleukin-15 protein” or “IL-15” as referred to herein includes any of the recombinant or naturally-occurring forms of the interleukin-15 (IL-15) protein or variants (also referred to herein as IL-15 agonist derivatives thereof) or homologs thereof that maintain IL-15 protein activity (e.g. within at least 50%, 80%, 90%, 95%, 96%, 97%, 98%, 99% or 100% activity compared to IL-15 protein). In embodiments, the derivatives or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 protein. In embodiments, the IL-15 protein is substantially identical to the protein identified by the UniProt reference number P40933 or a variant or homolog having substantial identity thereto.

An “interleukin-15 receptor subunit alpha protein” or “IL-15Rα” as referred to herein includes any of the recombinant or naturally-occurring forms of the interleukin-15 receptor subunit alpha (IL-15Rα) protein or variants/derivatives or homologs thereof that maintain IL-15Rα protein activity (e.g. within at least 50%, 80%, 90%, 95%, 96%, 97%, 98%, 99% or 100% activity compared to IL-15Rα protein). In embodiments, the variants/derivatives or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15Rα protein. In embodiments, the IL-15Rα protein is substantially identical to the protein identified by the UniProt reference number Q13261 or a variant/derivative or homolog having substantial identity thereto.

As used herein, “domain” refers to a conserved portion of a protein that functions and exists independently of the rest of the protein sequence. A domain may form a stable, three-dimensional structure that exists as a functional unit independent of the remaining protein. For example, the IL-15RαSu domain is the portion of IL-15Rα that retains the IL-15 binding activity.

As used herein, “IL-15 domain” refers to a polypeptide comprising at least a portion of a sequence of the IL-15 protein. In embodiments, the IL-15 domain comprises at least a portion of the sequence of the IL-15 protein and includes one or more amino acid substitutions or deletions within the amino acid sequence of the IL-15 protein. In embodiments, the IL-15 domain is an IL-15 variant that comprises a different amino acid sequence compared to the IL-15 protein. In embodiments, the IL-15 domain binds the IL-15Rα protein or a fragment thereof. In embodiments, the IL-15 domain is bound to the IL-15Rα protein or a fragment thereof. In embodiments, the sequence of the IL-15 domain has at least one amino acid change, e.g. substitution or deletion, compared to the IL-15 protein. In embodiments, the amino acid substitutions/deletions are in the portions of IL-15 that interact with IL-15Rβ and/or γC. In embodiments, the amino acid substitutions/deletions do not affect binding to the IL-15Rα polypeptide or the ability to produce the IL-15 domain. In embodiments, the amino acid substitutions/deletions increase or decrease binding affinity of IL-15 to the IL-15Rα domain, the IL-15Rβ domain, or the IL-15Rγ domain. In embodiments, amino acid substitutions can be conservative or non-conservative changes and insertions of additional amino acids compared to the IL-15 protein. In embodiments, the IL-15 domain comprises one or more than one amino acid substitutions/deletions at position 6, 8, 10, 61, 65, 72, 92, 101, 104, 105, 108, 109, 111, or 112 of the IL-15 protein sequence. In embodiments, the IL-15 domain comprises an N72D substitution of the IL-15 protein sequence.

The term “sushi domain” as used herein refers to a common motif in proteins comprising a beta-sandwich arrangement. Sushi domains are common in protein-protein interactions and typically include four cysteines forming two disulfide bonds in a 1-3 and 2-4 pattern. For example, the region of IL-15Ra that binds IL-15 includes a sushi domain.

In embodiments, the IL-15Rα sushi domain includes the amino acid sequence comprising the sequence of SEQ ID NO:1. In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to the sequence of SEQ ID NO:1. In embodiments, the IL-15Rα sushi domain associates with the IL-15 protein. In embodiments, the IL-15Rα sushi domain associates with the IL-15 domain.

(SEQ ID NO: 1)
Ile Thr Cys Pro Pro Pro Met Ser Val Glu His Ala
Asp Ile Trp Val Lys Ser Tyr Ser Leu Tyr Ser Arg
Glu Arg Tyr Ile Cys Asn Ser Gly Phe Lys Arg Lys
Ala Gly Thr Ser Ser Leu Thr Glu Cys Val Leu Asn
Lys Ala Thr Asn Val Ala His Trp Thr Thr Pro Ser
Leu Lys Cys Ile Arg

In embodiments, IL-15 or at least one member of an IL-15: IL-15Rα binding pair (covalently or non-covalently linked) is fused to a protein such as Ig or Tissue Factor, wherein the fusion confers pharmacokinetic stability on the IL-15 or IL-15: IL-15Rα. The IL-15, IL-15: IL-15Rα, or fusion protein derivative thereof retains binding affinity and agonist activity to IL-15Rβγ.

### N-803

N-803 (nogapendekin alfa inbakicept; also known as ALT-803; recombinant human super agonist interleukin-15 (IL-15) complex [also known as IL 15N72D:IL-15RαSu/lgG1 Fc complex])) is a soluble complex consisting of two protein subunits of a human IL-15 variant (nogapendekin alfa) bound with high affinity to a dimeric human IL-15Rα sushi domain/human IgG1 Fc fusion protein (inbakicept) (Han K P, et al. IL-15:IL-15 receptor alpha superagonist complex: high-level coexpression in recombinant mammalian cells, purification and characterization. Cytokine. 2011;56:804-10; Zhu X, et al. Novel human interleukin-15 agonists. J Immunol. 2009; 183:3598-607). The IL-15 variant (also referred to as an IL-15 agonist derivative) is a 114-amino acid polypeptide comprising the mature human IL 15 cytokine sequence with an Asn-to-Asp substitution at position 72 of helix C (N72D) (Zhu 2009). The human IL-15Rα sushi domain/human IgG1 Fc fusion protein comprises the sushi domain of the human IL-15 receptor a subunit (IL-15Rα) (amino acids 1-65 of the mature human IL-15Rα protein) linked with the human IgG1 CH2-CH3 region containing the Fc domain (232 amino acids). Aside from the N72D substitution, all the protein sequences are human. N-803 acts as a growth and activation factor for natural killer (NK) cells, and effector and memory T cells.

In any of the methods, uses or compositions disclosed herein, the IL-15 or IL-15 agonist derivative thereof is a stabilized fusion construct. In one aspect the IL-15 agonist derivative thereof is IL-15:IL-15Rα. In a preferred aspect the IL-15 derivative thereof is an IL-15N72D:IL-15RαSu/Fc complex (N-803), wherein the N-803 comprises a dimeric IL-15RαSu/Fc and two IL-15N72D molecules.

In one aspect, the IL-15 or IL-15 agonist derivative thereof, such as N-803, is administered systemically, for example, formulated in a pharmaceutically-acceptable buffer such as physiological saline. Preferable routes of administration include, for example, subcutaneous (SC), intravenous (IV), intraperitoneal, intramuscular, or intradermal injections that provide continuous, sustained levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin. In a preferred aspect, the administration route is via subcutaneous administration.

Compositions comprising IL-15 or an IL-15 agonist derivative thereof for parenteral use may be provided in unit dosage forms (e.g., in single-dose ampoules, syringes or bags), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Furthermore, the composition may include suspending, solubilizing, stabilizing, pH-adjusting agents, tonicity adjusting agents, and/or dispersing, agents.

As indicated above, the IL-15 or an IL-15 agonist derivative thereof and/or a composition comprising IL-15 or a derivative thereof may be in a form suitable for sterile injection.

Exemplary effective weight-based doses via SC administration of IL-15 or an IL-15 agonist derivative thereof, including N-803, include between 0.1 μg/kg and 50 μg/kg body weight, e.g., 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, or 50 μg/kg. In a preferred aspect, the dose is 10 μg/kg. Further, exemplary flat doses (SC administration) include between 0.4 mg and 5 mg (e.g., 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5., 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0 mg) is also acceptable, with a flat dose of 1.2 mg being preferred. As an example, a weight-based dose of 15 μg/kg SC has been safely used for numerous clinical trials of N-803 in advanced cancers, although a flat dose of 1.2 mg SC is also acceptable depending on study parameters. This dose was determined via conversion of the weight-based dose of 15 μg/kg using the average body weight of adults living in the US. Still further, doses administered intravesically include between 300 μg to 500 μg (e.g., 300, 305, 310, 315, 320, 325, 330, 335, 340, 345, 350, 355, 360, 365, 370, 375, 380, 385, 390, 395, 400, 405, 410, 415. 420, 425, 430, 435, 440, 445, 450, 455, 460, 465, 470, 475, 480, 485, 490 or 500 μg.

In one aspect, the patient is administered a dose every 14 days. In still another aspect, the dose is administered two or more times. In a preferred aspect, the patient is administered a first dose of the IL-15 or IL-15 agonist derivative thereof followed by a second dose 14 days after the first dose. In a preferred aspect, each dose is 10 μg/kg.

By the terms “effective amount” and “therapeutically effective amount” of a composition or formulation or formulation component is meant a sufficient amount of the composition, formulation or component, to provide the desired effect. For example, by “an effective amount” is meant an amount of a compound or a composition comprising a compound wherein the compound is IL-15 or an IL-15 agonist derivative thereof, such as N-803, required to ameliorate one or more symptoms associated with a viral infection relative to an untreated patient. In a preferred aspect, an effective amount of IL-15 or an IL-15 agonist derivative thereof, including N-803, is between 0.1 μg/kg and 50 μg/kg body weight administered subcutaneously to the patient.

In one aspect, the patient is further administered a SARS-CoV2 vaccine. In one aspect, the SARS-CoV2 vaccine comprises an Ad5[E-,E2b-] adenoviral vector comprising a nucleic acid sequence encoding a SARs-CoV-S protein and a nucleic acid sequence encoding a SARS-COV2 nucleocapsid protein (N) protein fused to an ETSD sequence. In one aspect, the SARS-CoV2 vaccine is administered to the patient as a prime and a boost vaccination.

In still another aspect, a non-specific immunoglobulin G (IgG) or crystabllizable (Fc) fragment thereof is co-administered to the patient, wherein the IgG or Fc fragment binds CD16 on the patient's NK cells. In one aspect, the binding of the CD16 on the patient's NK cells results in inhibition of NK cell mediated antibody dependent cellular cytotoxicity (ADCC).

As disclosed herein, patients having acute SARS-CoV2 viral infection and having at least two symptoms that have been present for at least 60 days (i.e. having Long Covid), detection of SARS-CoV2 plasma remnants is reduced and/or eliminated following administration of the IL-15 or IL-15 agonist derivative thereof. Further, these patients have an increase in NK cell frequencies following administration of the IL-15 or IL-15 agonist derivative thereof. Still further, these patients have increased SARS-CoV2 specific CD4 and CD*8+ T cell responses following administration of the IL-15 or IL-15 agonist derivative thereof. Still further, these patients have reduced SARS-CoV2 RNA in gut tissue following administration of the IL-15 or IL-15 agonist thereof.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLE

This example describes an open-label, single-arm exploratory treatment study. 20 participants received 2 doses of N-803 (each dose is 10 μg/kg) subcutaneously 14 days apart. The results demonstrate the safety and tolerability of subcutaneous administration of an IL-15 super agonist (N-803) in individuals who are experiencing Long COVID. Further, the ability of N-803 to affect virologic markers of chronic SARS-CoV2 infection, and the ability of N-803 to improve PASC outcomes, post-COVID NK cell counts and adaptive immune responses is demonstrated.

### Program Overview (LIINC Cohort)

This study occurs within the Long-term Impact of Infection with Novel Coronavirus (LIINC) cohort (Peluso M J, et al. Persistence, magnitude, and patterns of post-acute symptoms and quality of life following onset of SARS-CoV2 infection: cohort description and approaches for measurement. Open forum infectious diseases 2022: ofab640), an observational study of post-COVID conditions that has been ongoing at UCSF since April 2020. Since that time, LIINC has been characterizing the natural history and biology of LC. The cohort has recruited over 1,000 individuals, of whom over 400 report mild-to-severe LC symptoms. The retention rate at 1 year is 89%. Individuals are evaluated at scheduled visits and biological specimens (plasma, serum, peripheral blood mononuclear cells (PBMCs)) are collected and stored. A subset of the cohort is referred for additional studies including advanced cardiopulmonary (echocardiogram, cardiopulmonary exercise testing, cardiac magnetic resonance imaging (MRI), pulmonary function tests, and tilt table tests) neurologic (neuropsychiatric testing, lumbar puncture), imaging (fluorodeoxyglucose-positron emission tomography/computed tomography (FDG-PET/CT), and tissue assessment (gut biopsy, lymph node fine needle aspiration). As of April 2024, the LIINC team has conducted over 3000 participant-encounters and have enrolled over 200 individuals into the more intensive protocols. LIINC has also described the relationship between LC and adaptive immune responses (Peluso M J, et al. Long-term SARS-CoV2-specific immune and inflammatory responses in individuals recovering from COVID-19 with and without post-acute symptoms. Cell Rep 2021:109518), inflammation (Peluso M J, et al. Markers of Immune Activation and Inflammation in Individuals With Postacute Sequelae of Severe Acute Respiratory Syndrome Coronavirus 2 Infection. J Infect Dis 2021; Durstenfeld M S, et al. Role of antibodies, inflammatory markers, and echocardiographic findings in postacute cardiopulmonary symptoms after SARS-CoV2 infection. JCI Insight 2022;7; Peluso M J, et al. Plasma Markers of Neurologic Injury and Inflammation in People With Self-Reported Neurologic Postacute Sequelae of SARS-CoV2 Infection. Neurol Neuroimmunol Neuroinflamm 2022;9), autoimmunity (Peluso MJ, et al. Lack of Antinuclear Antibodies in Convalescent Coronavirus Disease 2019 Patients With Persistent Symptoms. Clin Infect Dis 2022;74:2083-4; Bodansky A, et al. Autoantigen profiling reveals a shared post-COVID signature in fully recovered and long COVID patients. JCI Insight 2023;8), microbial translocation (Giron LB, et al. Markers of fungal translocation are elevated during post-acute sequelae of SARS-CoV2 and induce NF-KB signaling. JCI Insight 2022;7), neurocognitive changes (Hellmuth J, et al. Persistent COVID-19-associated neurocognitive symptoms in non-hospitalized patients. J Neurovirol 2021;27:191-5; Apple AC, et al. Risk factors and abnormal cerebrospinal fluid associate with cognitive symptoms after mild COVID-19. Ann Clin Transl Neurol 2022;9:221-6), and cardiopulmonary physiology (Durstenfeld M S, et al. JCI Insight 2022;7; Durstenfeld M S, et al. Use of Cardiopulmonary Exercise Testing to Evaluate Long COVID-19 Symptoms in Adults: A Systematic Review and Meta-analysis. JAMA Netw Open 2022;5:e2236057).

Potential participants are required to enroll in LIINC, and the trial participants are recruited directly from LIINC if they have had LC subsequent to an acute COVID episode.

Study overview: this is a single-arm, open label study to assess the safety and efficacy of N-803 to improve immune function and to treat LC. Approximately 20 participants who meet the WHO LC criteria described herein are enrolled. Participants receive two subcutaneous doses of N-803 (10 μg/kg) subcutaneously into the abdomen 14 days apart (treatment Days 0 and 15).

### Criteria for Evaluation

### Efficacy Endpoints

The change in patient-reported outcomes (PROs) PROMIS-29 score from Baseline to D45 (30 days after final N-803 administration).

Change PROMIS-29 score in those with symptoms at Baseline and at D45, and D755555.

Change in other assessments (EuroQoL Quality of Life, neurocognitive testing, DASI, 6MWT performance) between Baseline and at D30 (2 weeks after last N-803 administration, D45, and D75).

Percentage of participants with no detection of SARS-CoV2 plasma remnants (i.e., viral detection by reverse transcriptase-polymerase chain reaction ((RT-PCR) and Spike protein fragments) compared to baseline at D45 and D75.

Change in frequency and activation of NK cells and SARS-CoV2 specific CD8+T cells from baseline to D45.

Proportion with reduction in inflammatory markers (e.g., interleukin-6 (IL-6), tumor necrosis factor alpha (TNF-alpha) at Baseline and at D45.

### Safety Evaluations

Number of participants with adverse events (AE)s (Treatment Emergent Adverse Events (TEAE)s, Serious Adverse Events (SAE)s, and Adverse Events of Special Interest (AESI)). The primary outcome will be the number of individuals in each group experiencing a severe (Grade 3 or greater) AE thought to be possibly, probably, or definitely related to study treatment throughout the study duration. Grade 3 local injection site reactions are expected and time to resolution will be followed closely (see TABLE 1).

TABLE 1
AE Severity Grading

| Severity | |
|---|---|
| (Toxicity | |
| Grade) | Description |
| Mild (1) | Transient or mild discomfort; no limitation in |
| | activity; no medical intervention or therapy |
| | required. The subject may be aware of the |
| | sign or symptom but tolerates it reasonably well. |
| Moderate (2) | Mild to moderate limitation in activity, no or |
| | minimal medical intervention/therapy required. |
| Severe (3) | Marked limitation in activity, medical intervention/ |
| | therapy required, hospitalizations possible. |
| Life-threatening (4) | The subject is at risk of death due to the adverse |
| | experience as it occurred. This does not refer to |
| | an experience that hypothetically might have caused |
| | death if it were more severe. |

### Subject Selection

Study Population: subjects with a history of SARS-CoV2 infection who meet the case definition for Long COVID, and who meet the inclusion and exclusion criteria are eligible for participation in this study.

### Inclusion Criteria

Age ≥18 and <70 years at Screening.

Enrolled or willing to enroll and complete at least 1 visit in the UCSF Long-term Impact of Infection with Novel Coronavirus (LIINC) study. Any adult who has been infected with SARS-CoV2 or has ever received or is eligible to receive a SARS-CoV2 vaccination, and who is able to provide written informed consent, is eligible to participate in LIINC.

History of at least one SARS-CoV2 infection, defined as report of a positive nucleic acid amplification test (NAAT) and/or a positive SARS-CoV2 antigen rapid diagnostic test (RDT). Written proof of the test will be requested but is not required as long as the participant attests to the positive test.

Clinical evidence of Long COVID (LC)

At least two symptoms or at least one severe symptom (such as those in the list below) that are new or worsened since the time of a SARS-CoV2 infection, not known to be attributable to another cause. At least two symptoms are present: systemic symptoms (e.g., fatigue, chills, post-exertional malaise), neurocognitive symptoms (e.g., trouble with memory/concentration (“brain fog”), headache, dysautonomia/postural orthostatic tachycardia syndrome, dizziness, unsteadiness, neuropathy, sleep disturbance), cardiopulmonary symptoms (e.g., chest pain, palpitations, shortness of breath, cough, fainting spells), musculoskeletal symptoms (e.g., muscle aches, joint pain), gastrointestinal symptoms (e.g., nausea, diarrhea). Although other symptoms (e.g., skin rash, hair loss, mental health symptoms, trouble with smell/taste, genitourinary symptoms) will be recorded and tracked, at least two core symptoms listed above must be present. The two symptoms can be from within the same category (for example, brain fog and headache).

Symptoms must have been present for at least 60 days prior to screening. Symptoms that wax and wane must have been initially present at least 60 days prior to screening. Symptoms must be reported to be at least somewhat bothersome and to have an impact on quality of life and/or everyday functioning. At least 90 days have elapsed since the most recent suspected or confirmed SARS-CoV2 infection and the time of screening. Not currently hospitalized. Body mass index (BMI) 18 to 50 kilograms/meter squared (kg/m2), inclusive, at the time of screening. In otherwise stable health, within 28 days prior to Screening, based on medical history, physical assessment, laboratory findings, and vital signs.

### Exclusion Criteria

Previously received SARS-CoV2 antiviral 30 days prior to planned Day 0 or plan to receive such treatment before exiting the study.

Plans to receive any investigational or approved vaccine or booster for SARS-CoV2 within 30 days prior to planned Day 0 or before Day 30 following planned Day 0.

Active cardiovascular disease, defined as known prior: myocardial infarction within 90 days of screening OR coronary artery bypass procedure within 90 days of screening OR current heart failure with reduced ejection fraction (<45%) OR current pulmonary arterial hypertension.

Known stroke within 3 months prior to planned Day 0. Known active bacterial, fungal, viral, or other infection besides SARS-CoV2 requiring treatment within the 28 days prior to Day 0 and meeting criteria for systemic involvement.

Major surgery within 6 months prior to planned Day 0 or planned major surgery during the first 180 days following planned Day 0.

History of unplanned hospitalization for >24 hours within 28 days prior to Screening.

Active Hepatitis B (Hep B) infection (defined as Hep B surface antigen (sAg) positive). Note: A known positive Hep B core antibody (cAb) in the absence of positive sAg is not considered exclusionary.

Active Hepatitis C (Hep C) infection (defined as Hep C Ab positive or indeterminate with detectable Hep C RNA). Note: Those with cured Hep C (Ab positive or indeterminate but negative Hep C RNA) will remain eligible.

Laboratory abnormalities including: ANC <1500 per mm³; platelet count <100,000 per mm³; baseline AST/ALT >1.5× ULN; hemoglobin <9 d/dL; CrCl <50 (estimated glomerular filtration rate); ALC<1000 per mm³; known or suspected HIV infection; end stage kidney disease requiring dialysis; history of Type 1 or Type 2 Diabetes mellitus requiring systemic medication or insulin; severe hepatic impairment (Child-Pugh Class C). Also included is moderate or severe immunocompromise, according to the current NIH COVID-19 Treatment Guidelines as of Mar. 6, 2023. The detailed list includes the following: (a) receiving active treatment for solid tumor or hematologic malignancy, including use of systemic chemotherapy for treatment of cancer within the year prior to screening, (b) has hematologic malignancies (e.g., chronic lymphocytic lymphoma, non-Hodgkin lymphoma, multiple myeloma, acute leukemia) and are known to have poor responses to COVID-19 vaccines or an increased risk of severe COVID-19, regardless of the treatment status for the hematologic malignancy, (c) received a solid-organ or islet transplant and is receiving active immunosuppressive therapy, (c) receiving chimeric antigen receptor T cell (CAR-T cell) therapy or hematopoietic cell transplant, on immunosuppressive therapy or transplant within the prior 2 years, (e) has moderate or severe primary immunodeficiency syndromes (e.g., severe combined immunodeficiency, DiGeorge syndrome, Wiskott-Aldrich syndrome, common variable immunodeficiency disease), (f) has advanced or untreated HIV infection (defined as people with HIV and CD4 T lymphocyte [CD4 ] cell counts <200 cells/mm³, a history of an AIDS-defining illness without immune reconstitution, or clinical manifestations of symptomatic HIV), (g receiving active high-dose corticosteroids (i.e., >=20 mg prednisone or equivalent daily per day for >=2 weeks), alkylating agents, antimetabolites, transplant-related immunosuppressive drugs, cancer chemotherapeutic agents classified as severely immunosuppressive, or immunosuppressive or immunomodulatory biologic agents (e.g., B cell—depleting agents). Further, known prior diagnosis of myalgic encephalomyelitis/chronic fatigue syndrome (ME/CFS), preceding and not related to SARS-CoV2 infection and not worsened since SARS-CoV2 infection. Known prior diagnosis of dysautonomia, preceding and not related to SARS-CoV-2 infection and not worsened since SARS-CoV2 infection. Known allergy to any components used in the formulation of the intervention. History of anaphylaxis or similar significant allergic reaction to prescription or non-prescription drugs or food products. Similarly, presence of severe atopic conditions represents significant risk for allergic reaction. Pregnant, breastfeeding, or unwilling to practice birth control. Participation in a clinical trial with receipt of an investigational product within 28 days prior to planned Day 0, with the exception of exploratory PET imaging studies related to Long COVID. Current alcohol or illicit drug use as determined by the investigator to preclude participation.

### Concurrent Medications

All subjects should be maintained on the same medications throughout the entire study period, as medically feasible, with no introduction of new chronic therapies unless deemed necessary by a medical provider with the exception of exclusionary medications as listed above. Initiation of over-the-counter supplements will be discouraged.

Allowed Medications and Treatments: participation in the study will not interfere with the participant's standard of care. Routine or standard of care vaccinations (such as influenza, pneumococcus, etc.) are allowed and will be documented in the CRFs; wherever possible these will be spaced greater than or equal to two weeks from study visits.

### Study Treatments

Method of Assigning Subjects to Treatment Groups: this is an open label, single-arm study given that study treatment is likely to lead to marked infusion site reactions and transient systemic signs and symptoms of immune activation (e.g., myalgias, fever).

Dosage/Dosage Regimen: N-803 is administered subcutaneously to the abdomen once every 14 days for a total of two doses. Subjects followed up 60 days later. Dosing in weight based and is 10 μg/kg of body weight.

Dispensing: subjects receive each of the subcutaneous doses during a study visit.

### Study Procedures and Guidelines

A Schedule of Events representing the required testing procedures to be performed for the duration of the study is diagrammed in Table 2 (at end of example section). The Schedule of Events will serve as the final resource for guiding study activities.

### Questionnaire-based Measurements

Long COVID Symptom Assessment. Signs and symptoms related to Long COVID are reviewed at all visits, using forms such as the LIINC CRFs. At D0 and all subsequent visits, all grades of signs and symptoms that are newly developed or changed since the previous visit are recorded. Duration (start and stop dates), severity/grade, outcome, treatment, and relation to study drug are recorded on the case report forms. All clinical events and new diagnoses or changes in diagnoses are recorded.

Other Symptom Assessment (AE Review). At D0 and all subsequent visits, all grades of signs and symptoms that are newly developed or changed since the previous visit are recorded. Symptoms not previously experienced as part of Long COVID, or which have worsened beyond the degree previously experienced by the participant, are considered to represent adverse events and recorded as such.

For these symptoms, duration (start and stop dates), severity/grade, outcome, treatment, and relation to study drug are recorded on the case report forms. All clinical events and new diagnoses or changes in diagnoses are recorded.

Quality of Life. Quality of life (QoL) is obtained using the EQ-5D-5L scale which is part of the LIINC CRFs. This includes the 100-point visual analogue scale (VAS).

The change in patient-reported outcomes (PROSs). The PROMIS-29 form is completed and scored 30 days following the last N-803 administration. This scale measures self-reported health, using a collection of short forms assessing fatigue, physical function, anxiety, depression, pain, sleep disturbance, and ability to participate in social roles and activities. The questionnaire uses a computer interface over approximately 5 minutes. This instrument has been used for ME/CFS and Long COVID. PROMIS-29 and change in other assessments (EuroQoL Quality of Life, neurocognitive testing, DASI, 6MWT performance) between Baseline and at 14, 30, and 60 days following last N- 803 administration.

Patient Global Impression of Change (PGIC). The self-report measure Patient Global Impression of Change (PGIC) reflects a patient's belief about the efficacy of their treatment. A modified PGIC scale is used which has been used to study pain syndromes and has been employed in other Long COVID clinical trials. It is a common data element developed by the National Institutes of Mental Health. Everyday Cognition Form (ECOG). The ECOG is performed in the parent LIINC study and asks participants to self-rate issues with cognition. It is a patient-reported measure for neurocognitive symptoms.

Neurocognitive Assessment. A neurocognitive assessment such as the NIH Toolbox or similar assessment is administered. Neurocognitive assessments assess motor, emotional, sensory, and cognitive function.

Duke Activity Status Index. The Duke Activity Status Index is a patient-reported estimate of functional capacity, maximal oxygen consumption (VO2 max) and maximum metabolic equivalent of tasks (METs). The DASI questionnaire produces a score between 0 and 58.2 points, which is linearly correlated with a patient's VO2 max and METs, as measured from cardiopulmonary exercise testing (CPET). It inquires about a person's ability to perform self-care, walk, climb stairs, run, do house and yard work, engage in sexual intercourse, and perform moderate recreational activities.

Dysautonomia Assessment (e.g., COMPASS-31 or similar scale). The Composite Autonomic Symptom Score (COMPASS)-31 is an assessment of autonomic dysfunction. This abbreviated questionnaire provides a quantitative measure of autonomic symptoms that otherwise might not be adequately recorded using the above instruments. It includes measures of orthostatic intolerance, vasomotor and secretomotor symptoms, GI and urinary symptoms, syncope, and genitourinary symptoms. This questionnaire or a similar instrument is used to determine the impact of treatment on post-COVID autonomic dysfunction.

DSQ-PEM (Short Form). The DePaul Symptom Questionnaire (DSQ) post-exertional malaise (PEM) questionnaire isused to assess post-exertional malaise at baseline, and again at the primary endpoint.

WHO-DAS (Short Form). The World Health Organization Disability Assessment Schedule 2.0 questionnaire asks about difficulties due to health conditions. Health conditions include diseases or illnesses, other health problems that may be short or long lasting, injuries, mental or emotional problems, and problems with alcohol or drugs. This questionnaire is used at baseline, and again at the primary endpoint.

6 Minute Walk Test. A 6MWT is performed.

Other evaluations include: percentage of participants with no detection of SARS-CoV2 plasma remnants (i.e., viral detection by reverse transcriptase-polymerase chain reaction ((RT-PCR) and Spike protein fragments) compared to baseline and at 14, 30, and 60 days post last N-803 administration; proportion with increased NK cell frequencies and SARS-CoV2-specific CD4 and CD8+ T cell responses 14, 30, and 60 days post last N-803 administration; percentage of participants with reduced SARS-CoV2 RNA in gut tissue approximately 30-60 days post administration; and proportion with reduction in inflammatory markers (e.g., interleukin-6 (IL-6), tumor necrosis factor alpha (TNF-alpha) at Baseline and 14 and 30 days post last N-803 administration.

Statistics-primary analysis plan: eligible patients who receive at least one dose of the study drug comprise the paired baseline-to 30 days after last N-803 administration time--points modified intent to treat (mITT) population and are the primary analysis population for all analyses. Exploratory analyses include mITT population with addition of efficacy evaluations at the 30 day post N-803 administration time point. Safety and tolerability data is summarized for all participants. Adverse event rates are coded by body system. Adverse events are tabulated by treatment group and include the number of patients for whom the event occurred, the rate of occurrence, and the severity and relationship to study drug. Adverse events between pre-and post-administration time points are summarized by proportions with associated 95% Clopper-Pearson confidence intervals and are compared using a two-sided 0.05 level Fisher exact test.

This is a study of Long COVID symptoms. As a result, symptoms experienced prior to receipt of the study intervention are documented, and a symptom will only be considered to represent an AE if its quality or severity differs from or exceeds that which was previously experienced by the participant.

Participants with Positive COVID Tests During the Study: due to the ongoing pandemic, it is anticipated that some individuals may test positive for COVID-19 during the course of the study. If an individual tests positive for SARS-CoV2 during the course of the study, existing testing and treatment programs are used to navigate the participant to care.

### Clinical Laboratory Measurements

Hematology: blood is obtained and sent to the clinical hematology lab for a complete blood count (hemoglobin, hematocrit, red blood cell count, white blood cell count, white blood cell differential, and platelet count), erythrocyte sedimentation rate (ESR), and serum C-reactive protein (CRP) determinations for assessment of systemic evidence for infection and/or inflammation.

Blood Chemistry Profile: blood is obtained and sent to the site's clinical chemistry lab for determination of serum sodium, potassium, chloride, bicarbonate, random glucose, BUN, creatinine, aspartate aminotransferase (AST/SGOT), alanine aminotransferase (ALT/SGPT), alkaline phosphatase, total bilirubin, direct bilirubin, albumin, phosphate, calcium, and complete blood counts (including ANC and ALC).

Pregnancy Testing: a urine or serum pregnancy test is obtained from female subjects who are of childbearing age prior to their participation in the study.

Hepatitis B Testing: subjects undergo Hep B surface antigen testing at Screening, if they have not had such screening within the preceding 6 months. Individuals with active Hep B are excluded. This is defined as detectable Hep B surface antigen. Individuals without detectable Hep B surface antigen remain eligible, regardless of their vaccination or antibody status (e.g., a positive Hep B cAb or negative Hep B sAb is not exclusionary).

Hepatitis C Testing: subjects undergo Hep C antibody testing at Screening, if they have not had such screening within the preceding 6 months. Individuals with active Hep C, defined as positive or indeterminate antibody with the presence of Hep C RNA, are excluded. Positive or indeterminate Hep C Ab in the absence of Hep C RNA is not exclusionary.

SARS-CoV2 PCR or Antigen Testing: swabs of the anterior nares is performed to assess for SARS-CoV2 shedding.

### Research Laboratory Measurements

Plasma Measures of Viral Persistence: plasma levels of SARS-CoV2 antigens (Spike, nucleocapsid, etc.) are performed to evaluate the effect of antiviral therapy on measures of viral persistence.

Immunophenotyping: single-or multi-plex platforms are used to measure targeted biomarkers previously shown to be elevated in Long COVID, which may include interleukin-6, TNF-alpha, monocyte chemoattractant protein-1, and interferon-gamma induced protein 10. In addition, high-throughput platforms such as Olink proteomics are used to conduct within-person and/or between-group comparisons to identify pathways that are altered with treatment, or that differ between those with and without improvement during the study period. Additional measures of cellular and humoral immunity are also performed. In addition, T cell and NK cell frequencies and phenotypes are measured by high-parameter spectral flow cytometry and SARS-CoV2 N and S-specific CD8+ and CD4+ T cell frequencies are determined by intracellular cytokine measurement following antigen stimulation.

SARS-CoV2 Genotyping: genotyping is performed on any samples collected during the study in which SARS-CoV2 virus is recovered. Participants with a positive test at the time of screening are not eligible for the study. subjects with suspected acute SARS-CoV2 infection or who report rebound symptoms at the time of an in-person follow-up visit will undergo a SARS-CoV2 test and if positive the sample will be banked for genotyping analyses.

### Evaluations by Visit

The details of the visit schedule are outlined below. The Schedule of Events will be the final guide for what events are to occur at each specific study visit.

### Screening Visit (D-60)

Following the informed consent discussion and signed informed consent form, the Screening Assessment will be performed. The inclusion and exclusion criteria are reviewed with the participant. Screening labs, medical history, and the details of concomitant medications are obtained. Details of the participant's COVID-19 history (including COVID-19 treatments) are reviewed and/or confirmed.

The Screening Assessment includes a physical assessment by a study clinician (physician, nurse, or physician assistant). Other assessments as outlined in the Schedule of Events will be performed.

Laboratory tests as outlined in the Schedule of Events is performed. Laboratory tests or other assessments performed for a clinical indication (not exclusively to determine study eligibility) is used for screening values even if the studies were performed before informed consent was obtained. An individual who recently had laboratory tests performed as part of routine clinical care need not repeat these tests if they are within the window for the test (e.g., within 6 months for hepatitis testing). For example, someone with recent Hepatitis B or C or HIV testing may not need to have the test repeated, as indicated in the Schedule of Events.

Screening evaluations should be completed within 28 days of planned D0. If more than 28 days lapse, screening procedures are repeated to re-confirm eligibility prior to D0. At a minimum, this will include the metabolic panel, complete blood count, and pregnancy testing. Laboratory tests that are not expected to change (such as coagulation studies or viral hepatitis status) are not required to be repeated.

### Baseline Evaluation (D-21 to D0)

An additional visit is performed following screening, at least 7 days after the screening visit and prior to D0. The target date for this visit is within 7 days prior to the scheduled date of study product administration, although up to 21 days is acceptable. The purpose of this visit is to make additional assessments prior to the intervention, to establish baseline symptomatology and measurements. This includes a symptom assessment and various clinical assessments as outlined in the Schedule of Events. Certain real-time laboratory tests are obtained to establish a baseline.

Note, after biospecimen collection has occurred, the participant may immediately move on to the intervention visit (medications can be dispensed after the baseline evaluations are complete).

### Intervention (D0)

Participants who screen into the study and complete baseline measurements are administered N-803.

Telephone Follow-ups (Phone Follow-Up, D1, D4, D16, D20): the subject is contacted by telephone to check on the overall response to each dose of medication. The Phone FU D1 visit focuses on determining whether there have been any adverse events from the study product. The Phone FU on D4 focuses on determining whether there have been any adverse events from the study product, as well as whether there has been any short-term efficacy.

In Person Follow-up Visits (D7, D15, D30, D45, D75): in-person follow-up visits occur as outlined in the Schedule of Events. At each visit, vital signs are collected, and the participant undergos a targeted physical assessment as needed. They will complete clinical assessments as outlined in the Schedule of Events. Laboratory tests are ordered and biospecimens collected and stored for later testing as per the Schedule of Events. The second dose of N-803 will be administered on D15.

Telephone or In Person End-Of-Study Visit (EOS D75): the subject is contacted by telephone or by in person interview with study staff to undergo clinical questionnaire assessments as outlined in the Schedule of Events. Unscheduled (Interim) Visits

### Statistical Methods and Considerations

Prior to the analysis of the final study data, a detailed Statistical Analysis Plan (SAP) is written describing all analyses that will be performed. The SAP contains any modifications to the analysis plan described below.

### Data Sets Analyzed

Eligible patients who are randomized into the study and receive at least one dose of the study drug during the randomized stage of this study comprise the modified intent to treat (mITT) population and are the primary analysis population for all analyses.

Per protocol analysis is based on description of the results per study group excluding anyone who has received a non-study COVID antiviral therapy during the trial period.

### Demographic and Baseline Characteristics

The following demographic variables at screening will be summarized by treatment group.

### Analysis of Primary Endpoint

Safety and tolerability data is summarized by treatment group.

Adverse event rates are coded by body system. Adverse events are tabulated by treatment group and include the number of patients for whom the event occurred, the rate of occurrence, and the severity and relationship to study drug. Adverse events between groups are summarized by proportions with associated 95% Clopper-Pearson confidence intervals and are compared using a two-sided 0.05 level Fisher exact test.

### Analysis of Secondary Endpoints

The key secondary outcome is the change in the PROMIS-29 scores from baseline to D10 follow up. This is analyzed from the mITT population using a linear regression model for follow-up PROMIS-29 with terms for the baseline value, randomized treatment and randomization strata (<26 v. >=26 weeks of symptoms)—an analysis of covariance model (ANCOVA) and tested using a two-sided 0.05 level test.

Continuous secondary events (e.g., IL-6) is compared using the ANCOVA approach. Antigen detection is summarized by proportion with 95% Clopper-Pearson confidence intervals and is compared using a two-sided 0.05 level Fisher exact test.

Primary and secondary endpoint analyses is performed on the mITT of the treatment group (single arm).

### Sample Size and Randomization

20 participants who meet the WHO LC criteria are enrolled.

For PROMIS-29, assuming a standard deviation (SD) of the within-person difference of 10, then the planned sample size has80% power to detect a difference of 11.2 on a two-sided 0.05 level test.

For biomarkers, such as IL-6, a difference from a mean value of 2.5 pg/mL to times the within-person standard deviation with 80% power is able to be detected.

It is assumed that 25% of participants have positive antigen at entry. There is a 62% power to detect a reduction to <1% with positive antigen on the treatment arm.

### REFERENCES

1. Robertson M M, Qasmieh S A, Kulkarni S G, et al. The Epidemiology of Long Coronavirus Disease in US Adults. Clin Infect Dis 2023;76:1636-45.
2. Nalbandian A, Sehgal K, Gupta A, et al. Post-acute COVID-19 syndrome. Nat Med 2021.
3. Global Burden of Disease Long CC, Wulf Hanson S, Abbafati C, et al. Estimated Global Proportions of Individuals With Persistent Fatigue, Cognitive, and Respiratory Symptom Clusters Following Symptomatic COVID-19 in 2020 and 2021. JAMA 2022;328:1604-15.
4. Proal D, VanElzakker M B, Aleman S, et al. SARS-CoV2 reservoir in post-acute sequelae of COVID-19 (PASC). Nat Immunol 2023;24:1616-27.
5. Peluso M J, Anglin K, Durstenfeld M S, et al. Effect of Oral Nirmatrelvir on Long COVID Symptoms: 4 Cases and Rationale for Systematic Studies. Pathog Immun 2022;7:95-103.
6. Gaebler C, Wang Z, Lorenzi J C C, et al. Evolution of antibody immunity to SARS-CoV2. Nature 2021.
7. Aydillo T, Gonzalez-Reiche A S, Aslam S, et al. Shedding of Viable SARS-CoV2 after Immunosuppressive Therapy for Cancer. N Engl J Med 2020;383:2586-8.
8. Choi B, Choudhary M C, Regan J, et al. Persistence and Evolution of SARS-CoV2 in an Immunocompromised Host. N Engl J Med 2020;383:2291-3.
9. Zollner A, Koch R, Jukic A, et al. Postacute COVID-19 is Characterized by Gut Viral Antigen Persistence in Inflammatory Bowel Diseases. Gastroenterology 2022; 163:495-506.e8.

110. Natarajan A, Zlitni S, Brooks E F, et al. Gastrointestinal symptoms and fecal shedding of SARS-CoV-2 RNA suggest prolonged gastrointestinal infection. Med (N Y) 2022.
11. Xu Q, Milanez-Almeida P, Martins A J, et al. Adaptive immune responses to SARS-CoV2 persist in the pharyngeal lymphoid tissue of children. Nat Immunol 2023;24:186-99.
12. Cheung C C L, Goh D, Lim X, et al. Residual SARS-CoV2 viral antigens detected in GI and hepatic tissues from five recovered patients with COVID-19. Gut 2022;71:226-9.
13. Goh D, Lim J C T, Fernandez S B, et al. Case report: Persistence of residual antigen and RNA of the SARS-CoV2 virus in tissues of two patients with long COVID. Front Immunol 2022; 13:939989.
14. Stein S R, Ramelli S C, Grazioli A, et al. SARS-CoV2 infection and persistence in the human body and brain at autopsy. Nature 2022;612:758-63.
15. Peluso M J, Ryder D, Flavell R, et al. Multimodal Molecular Imaging Reveals Tissue-Based T Cell Activation and Viral RNA Persistence for Up to 2 Years Following COVID-19. medRxiv 2023.
16. Jin J C, Ananthanarayanan A, Brown JA, et al. SARS CoV-2 detected in neonatal stool remote from maternal COVID-19 during pregnancy. Pediatr Res 2022:1-8.
17. Racine T, Kobinger GP. Viral pathogenesis: Unlocking Ebola persistence. Nat Microbiol 2017;2:17124.
18. Tejerina F, Catalan P, Rodriguez-Grande C, et al. Post-COVID-19 syndrome. SARS-CoV2 RNA detection in plasma, stool, and urine in patients with persistent symptoms after COVID-19. BMC Infect Dis 2022;22:211.
19. Swank Z, Senussi Y, Manickas-Hill Z, et al. Persistent circulating SARS-CoV2 spike is associated with post-acute COVID-19 sequelae. Clin Infect Dis 2022.
20. Peluso M J, Kelly J D, Lu S, et al. Persistence, magnitude, and patterns of postacute symptoms and quality of life following onset of SARS-CoV2 infection: cohort description and approaches for measurement. Open forum infectious diseases2022:ofab640.
21. Peluso M J, Deitchman A N, Torres L, et al. Long-term SARS-CoV2-specific immune and inflammatory responses in individuals recovering from COVID-19 with and without post-acute symptoms. Cell Rep 2021:109518.
22. Peluso M J, Lu S, Tang A F, et al. Markers of Immune Activation and Inflammation in Individuals With Postacute Sequelae of Severe Acute Respiratory Syndrome Coronavirus 2 Infection. J Infect Dis 2021.
23. Durstenfeld M S, Peluso M J, Kelly J D, et al. Role of antibodies, inflammatory markers, and echocardiographic findings in postacute cardiopulmonary symptoms after SARS-CoV2 infection. JCI Insight 2022;7.
24. Peluso M J, Sans H M, Forman C A, et al. Plasma Markers of Neurologic Injury and Inflammation in People With Self-Reported Neurologic Postacute Sequelae of SARS-CoV2 Infection. Neurol Neuroimmunol Neuroinflamm 2022;9.
25. Peluso M J, Thomas I J, Munter S E, Deeks S G, Henrich T J. Lack of Antinuclear Antibodies in Convalescent Coronavirus Disease 2019 Patients With Persistent Symptoms. Clin Infect Dis 2022;74:2083-4.
26. Bodansky A, Wang C Y, Saxena A, et al. Autoantigen profiling reveals a shared post-COVID signature in fully recovered and long COVID patients. JCI Insight 2023;8.
27. Giron L B, Peluso M J, Ding J, et al. Markers of fungal translocation are elevated during post-acute sequelae of SARS-CoV2 and induce NF-KB signaling. JCI Insight 2022;7.
28. Hellmuth J, Barnett T A, Asken B M, et al. Persistent COVID-19-associated neurocognitive symptoms in non-hospitalized patients. J Neurovirol 2021;27:191-5.
29. Apple A C, Oddi A, Peluso M J, et al. Risk factors and abnormal cerebrospinal fluid associate with cognitive symptoms after mild COVID-19. Ann Clin Transl Neurol 2022;9:221-6.
30. Durstenfeld M S, Sun K, Tahir P, et al. Use of Cardiopulmonary Exercise Testing to Evaluate Long COVID-19 Symptoms in Adults: A Systematic Review and Meta-analysis. JAMA Netw Open 2022;5:e2236057.

TABLE 2
SCHEDULE OF EVENTS

| | Day | | | | | | | | | | | |
|---|---|---|---|---|---|---|---|---|---|---|---|---|
| | −60 to −7 | −21 to 0 | 0 | 1 | 4 | 7 | 15 | 16 | 20 | 30 | 45 | 75 |
| | Visit Name | | | | | | | | | | | |
| | | | | | | | | | Phone | | | |
| | | | D 0 | Phone | Phone | | D 15 | Phone | FU | | D 45 | |
| | Screen¹ | Baseline¹ | N-803² | FU D 1 | FU D 4 | D 7 | N-803 | FU D 16 | D 20 | D 30 | P.E.E. | EOS |
| | Visit Window | | | | | | | | | | | |
| | N/A | N/A | N/A | +1 | +1/−1 | +2/−2 | +3/−3 | +1 | +1/−1 | +3/−3 | +3/−3 | +/−14 |
| Screening | | | | | | | | | | | | |
| Procedures | | | | | | | | | | | | |
| Informed | x | | | | | | | | | | | |
| Consent | | | | | | | | | | | | |
| Eligibility | x | | | | | | | | | | | |
| Review | | | | | | | | | | | | |
| Screening | x | | | | | | | | | | | |
| Labs | | | | | | | | | | | | |
| Screening | x | | | | | | | | | | | |
| Pregnancy | | | | | | | | | | | | |
| Test | | | | | | | | | | | | |
| Demographics | x | | | | | | | | | | | |
| Medical | x | | | | | | | | | | | |
| History | | | | | | | | | | | | |
| COVID-19 | x | | | | | | | | | | | |
| History | | | | | | | | | | | | |
| Concomitant | x | x | x | x | x | x | x | x | x | x | x | x |
| Medications | | | | | | | | | | | | |
| Study Product | | | | | | | | | | | | |
| Administration | | | | | | | | | | | | |
| N-803 | | | x | | | | x | | | | | |
| subcutaneous | | | | | | | | | | | | |
| Clinical | | | | | | | | | | | | |
| Assessments | | | | | | | | | | | | |
| Vital Signs | x | x | x | | | x | x⁴ | | | x | x | x |
| Physical | x | PRN | PRN | | | PRN | PRN | | | PRN | PRN | |
| Assessment | | | | | | | | | | | | |
| Long COVID | x | x | x | | x³ | x | x | | x³ | x | x | x³ |
| Symptom | | | | | | | | | | | | |
| Assessment | | | | | | | | | | | | |
| Quality of Life | x | x | x | | | | x | | | x | x | x³ |
| PROMIS-29 | x | x | | | | | x | | | x | x | x |
| PGIC Scale | | | | | | x | x | | | x | x | x |
| Duke Activity | x | x | | | | | x | | | x | x | x |
| Status Index | | | | | | | | | | | | |
| Dysautonomia | | x | | | | | x | | | x | x | x |
| Assessment | | | | | | | | | | | | |
| ECOG (LIINC) | | x | | | | | x | | | x | x | x |
| DSQ-PEM | | x | | x | | | x | x | | x | x | x |
| Neurocognitive | | x | | | | | x | | | x | x | x |
| assessment | | | | | | | | | | | | |
| 6MWT | | x | | | | | x | | | x | x | |

Safety Outcomes

| | | | | | | | | | | | | |
|---|---|---|---|---|---|---|---|---|---|---|---|---|
| AE Review | x | x | x | ×³ | ×³ | x | x | x³ | x³ | x | x | x |
| Safety Labs | | | x | | | x | x | | | x | x | |

Real-time laboratory tests

| | | | | | | | | |
|---|---|---|---|---|---|---|---|---|
| Pregnancy | x | | x⁵ | | x⁵ | | | |
| testing | | | | | | | | |
| Complete | x | | x | | x | | | |
| metabolic | | | | | | | | |
| panel | | | | | | | | |
| CBC/diff | x | | x | | x | | | |
| Hep B surface | x | | | | | | | |
| antigen | | | | | | | | |
| Within 6 | | | | | | | | |
| months of | | | | | | | | |
| Screen | | | | | | | | |
| Hep C | x | | | | | | | |
| antibody | | | | | | | | |
| Within 6 | | | | | | | | |
| months of | | | | | | | | |
| Screen | | | | | | | | |
| Hep C RNA (if | x | | | | | | | |
| Ab positive) | | | | | | | | |
| Within 6 | | | | | | | | |
| months of | | | | | | | | |
| Screen | | | | | | | | |
| Plasma HIV | x | | | | | | | |
| Ab/antigen | | | | | | | | |
| combination | | | | | | | | |
| testing | | | | | | | | |
| Within 2 | | | | | | | | |
| months of | | | | | | | | |
| Screen | | | | | | | | |
| C-reactive | | x | x⁶ | | x | | x | |
| protein | | | | | | | | |
| SARS-CoV-2 | x | PRN | PRN | PRN | PRN | PRN | PRN | PRN |
| PCR or Ag test | | | | | | | | |

Post-hoc laboratory tests

| | | | | | | | | |
|---|---|---|---|---|---|---|---|---|
| Biospecimen | x | x | x⁶ | x | x | x | x | x |
| storage | | | | | | | | |
| Markers of | x | x | x⁶ | x | x | x | x | x |
| viral | | | | | | | | |
| persistence | | | | | | | | |
| Markers of | | x | x⁶ | x | x | x | x | x |
| inflammation, | | | | | | | | |
| immune | | | | | | | | |
| phenotyping | | | | | | | | |

²The baseline visit and intervention visit may be combined to minimize participant burden.
³Assessments at the D 1 Phone FU focus on adverse events since taking the medication. Assessments at the D 4 Phone FU focus on the preceding 2 days (rather than the preceding month) to assess AEs and the potential impact of treatment.
⁴Clinical assessments and labs performed prior to N-803 administration.
⁵For persons of childbearing potential, a negative pregnancy test must be confirmed prior to the participant being dosed with the medication.
⁶If the intervention visit is on the same day as the baseline visit, blood collected for biospecimen storage is collected once.

What is claimed is:

1. A method of treating one or more symptoms associated with a viral infection in a patient exposed to a virus, wherein the virus is no longer detectable in the patient, and wherein the symptoms develop or persist after clearance of the virus, the method comprising administering to the patient an effective amount of a pharmaceutical composition comprising interleukin-15 (IL-15) or an IL-15 agonist derivative thereof.

2. The method of claim 1, wherein the symptoms are maintained for longer than 4 weeks after the patient has been diagnosed with the viral infection and wherein the viral infection is acute SARS-CoV2 infection.

3. The method of claim 1, wherein the symptoms are selected from the group consisting of neurological symptoms, cardiovascular symptoms, cardiopulmonary symptoms, gastro- intestinal symptoms, musculoskeletal symptoms, systemic symptoms and combinations thereof.

4. The method of claim 1, wherein the virus is a coronavirus.

5. The method of claim 4, wherein the coronavirus is SARS-CoV2.

6. The method of claim 2, wherein patient positivity for SARS-CoV2 viral infection exceeds 14 days.

7. The method of claim 1, wherein a non-specific immunoglobulin G (IgG) or fragment crystallizable (Fc) fragment thereof is co-administered to the patient, wherein the IgG or Fc fragment binds to CD16 on the patient's natural killer (NK) cells.

8. The method of claim 1, wherein the IL-15 agonist derivative is a stabilized fusion construct.

9. The method of claim 1, wherein the IL-15 agonist derivative is IL-15:IL-15Rα.

10. The method of claim 1, wherein the IL-15 agonist derivative is nogapendekin alpha imbakicept (N-803).

11. The method of claim 1, wherein the IL-15 or II-15 agonist derivative thereof is administered subcutaneously.

12. The method of claim 11, wherein the IL-15 or IL-15 agonist derivative thereof is administered every 14 days.

13. The method of claim 11, wherein the IL-15 or IL15 agonist derivative thereof is administered two or more times.

14. The method of claim 11, wherein the IL-15 or IL-15 agonist derivative thereof is administered at 1-50 μg/kg body weight.

15. The method of claim 3, wherein the patient has at least two symptoms and the symptoms have been present for at least 60 days.

16. The method of claim 1, further comprising administering a SARS-CoV2 vaccine.

17. The method of claim 16, wherein the SARS-CoV2 vaccine is administered as a prime and a boost vaccination.

18. The method of claim 1, wherein detection of SARS-CoV2 plasma remnants is reduced and/or eliminated following administration of the IL-15 or IL-15 agonist derivative thereof.

19. The method of claim 1, wherein the patient has increased NK cell frequencies following administration of the IL-15 or IL-15 agonist derivative thereof.

20. The method of claim 1, wherein the patient has increased SARS-CoV2 specific CD4 and CD8+ T cell responses following administration of the IL-15 or IL-15 agonist derivative thereof.

21. The method of claim 1, wherein the patient has reduced SARS-CoV2 RNA in gut tissue following administration of the IL-15 or IL-15 agonist derivative thereof.
